(19) United States
(12) Patent Application Publication
(10) Pub. No.: US 2003/0157047 A1
(43) Pub. Date: Aug. 21, 2003

(54) Cosmetic composition for removing make-up from and clening the skin

(75) Inventors: Paula Lennon, Lyon (FR); Cecile Boschet, Chevilly-Larue (FR); Carole Guiramand, Jouy-En-Josas (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/270,331

(22) Filed: Oct. 15, 2002

Related U.S. Application Data
(60) Provisional application No. 60/411,374, filed on Sep. 18, 2002.

(30) Foreign Application Priority Data
Oct. 15, 2001 (FR) 0113271
Aug. 22, 2002 (FR) 0210494

(51) Int. Cl. A61K 7/06; A61K 7/11

(52) U.S. Cl. 424/70.11; 424/70.21

(57) ABSTRACT

The invention relates to composition containing an oil-in-water emulsion and (1) an amphiphilic polymer containing polymerized units of at least one monomer comprising ethylenic unsaturation comprising a sulphonic group, in the free or partially or completely neutralized form, and containing at least one hydrophobic part, and (2) at least one make-up-removing oil.

The composition according to the invention can be used in particular for removing make-up from and/or cleaning the skin, lips and/or eyes.

This application claims priority to U.S. provisional application No. 60/411,374 (Attorney Docket 228066USOPROV) filed Sep. 18, 2002 and French patent applications 0210494 filed Aug. 22, 2002 and 0113171 filed Oct. 15, 2001, all incorporated herein by reference.

### BACKGROUND OF THE INVENTION

1. Field of the Invention

The invention relates to a make-up-removing and/or cleaning composition which is provided in the form of an oil-in-water emulsion comprising a specific amphiphilic polymer and at least one make-up-removing oil. This composition may be used in particular for removing make-up from and/or cleaning the skin, lips and/or eyes.

2. Discussion of the Background

Make-up removers and cleaners for the face provided in the form of emulsions, used conventionally to date, exhibit disadvantages which depend on the sense of the emulsion. In the case of water-in-oil emulsions, which are very efficient for make-up removing/cleaning owing to the fact that the fatty phase is directly available to dissolve the various fatty substances present on the skin or resulting from the make-up, their use is uncomfortable on account of the greasy and heavy feeling contributed by this external fatty phase, which remains on the skin.

In the case of oil-in-water (O/W) emulsions, the external aqueous phase contributes freshness. On the other hand, as the oily phase is the internal phase, it is not directly available to dissolve the fatty substances and, for this reason, make-up removal is less effective. In point of fact, for cleaning the face and more especially for removing make-up, which consists of removing all the make-up products, women are looking to obtain make-up removal which is as effective as possible.

To improve the effectiveness of a make-up-removing composition in the form of an O/W emulsion, it is necessary to introduce a significant amount of fatty phase (oily phase), since it is the latter which provides good dissolution of the solid particles of which the make-up is composed. However, when a large amount of oil is introduced into an aqueous phase gelled by a conventional aqueous gelling agent, the stability of the composition is found to be compromised beyond a certain amount of oil and it is then necessary to add in an emulsifying surfactant which, present in a sufficient amount, will stabilize the emulsion.

However, for reasons of harmlessness, in particular when the composition is used for removal of make-up from the eyes, it is important to use emulsions which do not comprise emulsifying surfactants. To ensure the stability of emulsions without an emulsifying surfactant, it is known to gel the external aqueous phase. However, when a large amount of oil is introduced into an aqueous phase gelled by a conventional aqueous gelling agent, the stability of the composition is found to be compromised beyond a certain amount of oil if, in addition, an emulsifying surfactant is not added. And the more fluid the medium, the more difficult it is to prepare emulsions without an emulsifying surfactant with a high level of oil.

Furthermore, while some gelling agents, such as crosslinked acrylic copolymers (Pemulen for example), make it possible to emulsify more than 20% by weight of oil, the compositions obtained have mediocre cosmetic properties as the feel thereof is heavy and sticky and thus not much appreciated by users.

### OBJECTS OF THE INVENTION

The need consequently exists for a cleaning and/or make-up-removing composition in the form of an oil-in-water emulsion which is stable and effective while not comprising an emulsifying surfactant and while having good cosmetic properties.

### SUMMARY OF THE INVENTION

The Inventors have now discovered a formulation using an amphiphilic polymer which makes it possible to achieve these objectives, this being the case whatever the viscosity of the composition, that is to say whether it is fluid or thick.

### DETAILED DESCRIPTION OF THE INVENTION

The present invention thus relates to a cleaning and/or make-up-removing cosmetic composition comprising an oily phase dispersed in an aqueous phase, wherein it comprises (1) an amphiphilic polymer comprising at least one monomer comprising ethylenic unsaturation comprising a sulphonic group, in the free or partially or completely neutralized form, and comprising at least one hydrophobic part and (2) at least one make-up-removing oil.

In addition, it has been found that, when the composition comprises at least 40% by weight of oily phase with respect to the total weight of the composition, it is preferable to use noncrosslinked amphiphilic polymers comprising at least two hydrophobic side parts (that is to say, two hydrophobic side chains).

Consequently, another of the present invention is a cleaning and/or make-up-removing composition comprising an oily phase dispersed in an aqueous phase, wherein it comprises at least one noncrosslinked amphiphilic polymer comprising at least one monomer comprising ethylenic unsaturation comprising a sulphonic group, in the free or partially or completely neutralized form, comprising at least two hydrophobic side parts, and in that the oily phase comprises at least one make-up-removing oil and constitutes at least 40% by weight with respect to the total weight of the composition.

As the invention composition is in a way a cosmetic composition, it preferably comprises a physiologically acceptable medium. The term “physiologically acceptable medium” is understood to mean, in the present application, a medium compatible with any keratinous substance, such as the skin, including the scalp, the nails, the mucous membranes, the eyes and the hair or any other skin region of the body.

The composition of the invention, even devoid of any emulsifying agent, exhibits excellent stability, whatever its viscosity and whatever the content of oils, and, in addition, exhibits very good make-up-removing effectiveness. Furthermore, the cosmetic feel of the composition according to the invention is very pleasant: there is no sticky effect and it is fresh during application to the skin.

The composition according to the invention preferably exhibits a viscosity ranging from 0.1 Pa.s (i.e. 1 poise) to 23 Pa.s (230 poises), preferably from 0.1 Pa.s (1 poise) to 15 Pa.s (150 poises) and better still from 0.2 to 15 Pa.s (2 to 150 poises), this viscosity being measured at ambient temperature (25° C.) with the RM180 Rheomat device from Mettler.

The composition according to the invention generally has a pH ranging from 3 to 9, better still from 4 to 9, and better still from 4.5 to 8 and even better still from 6 to 8.

Amphiphilic Polymers

The polymers used in accordance with the invention are amphiphilic polymers comprising at least one monomer comprising ethylenic unsaturation comprising a sulphonic group, in the free or partially or completely neutralized form, and comprising at least one hydrophobic part.

The term “amphiphilic polymer” is understood to mean any polymer comprising both a hydrophilic part and a hydrophobic part, and in particular a fatty chain.

A polymer used in the composition of the invention is a hydrophilic polymer. The term “hydrophilic polymer” is understood to mean any polymer which is soluble or dispersible in water, as such or in the neutralized form.

The hydrophobic part present in the polymers of the invention preferably comprises from 6 to 50 carbon atoms, more preferably from 6 to 30, better still from 6 to 22, carbon atoms, more preferably still from 6 to 18 carbon atoms and more particularly from 12 to 18 carbon atoms.

The polymers used according to the invention are amphiphilic polymers capable of being obtained from at least one monomer comprising ethylenic unsaturation comprising a sulphonic group, in the free or partially or completely neutralized form, and from at least one hydrophobic monomer.

Preferably, the polymers in accordance with the invention are partially or completely neutralized by a cosmetically acceptable base which can be an inorganic base (e.g., sodium hydroxide, potassium hydroxide or ammonia) or an organic base, such as mono-, di- or triethanolamine, an aminomethylpropanediol, N-methylglucamine, basic amino acids, such as arginine and lysine, and mixtures of these compounds.

The amphiphilic polymers in accordance with the invention are not limited in molecular weight, but preferably have a number-average molecular weight ranging from 1 000 to 20 000 000 g/mol, more preferably ranging from 20 000 to 5 000 000 and more preferably still from 100 000 to 1 500 000 g/mol.

The amphiphilic polymers according to the invention can be crosslinked or noncrosslinked.

The term “noncrosslinked polymer” is understood to mean a polymer which has not reacted with crosslinking agents and which therefore does not comprise crosslinking groups. It can be a linear or branched polymer. The noncrosslinked polymer used in the composition of the invention is preferably linear.

When the polymers are crosslinked, the crosslinking agents can be any known and preferably are selected from the group consisting of the compounds comprising olefinic polyunsaturation commonly used for the crosslinking of the polymers obtained by free-radical polymerization.

Mention may be made, for example, of divinylbenzene, diallyl ether, dipropylene glycol diallyl ether, polyglycol diallyl ethers, triethylene glycol divinyl ether, hydroquinone diallyl ether, ethylene glycol di(meth)acrylate, tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allyl ethers of alcohols of the sugar series, or other allyl or vinyl ethers of polyfunctional alcohols, and allyl esters of derivatives of phosphoric and/or vinylphosphonic acid, or the mixtures of these compounds.

According to a preferred embodiment of the invention, the crosslinking agent is selected from the group consisting of methylenebisacrylamide, allyl methacrylate or trimethylolpropane triacrylate (TMPTA). The degree of crosslinking is not limited and preferably varies from 0.01 to 10 mol % and more particularly from 0.2 to 2 mol % with respect to the polymer.

The monomers comprising ethylenic unsaturation comprising a sulphonic group used to make the invention amphiphilic polymers are not limited and preferably are chosen in particular from vinylsulphonic acid, styrenesulphonic acid, (meth)acrylamido(C₁-C₂₂)alkylsulphonic acids, N-(C₁-C₂₂)alkyl(meth)acrylamido(C₁-C₂₂)alkylsulphonic acids, such as undecylacrylamidomethanesulphonic acid, and their partially or completely neutralized forms, and their mixtures.

According to a preferred embodiment of the invention, use is made, as monomers comprising ethylenic unsaturation comprising a sulphonic group, of (meth)acrylamido(C₁-C₂₂)alkylsulphonic acids, such as, for example, acrylamidomethanesulphonic acid, acrylamidoethanesulphonic acid, acrylamidopropanesulphonic acid, 2-acrylamido-2-methylpropanesulphonic acid, 2-methacrylamido-2-methylpropanesulphonic acid, 2-acrylamido-n-butanesulphonic acid, 2-acrylamido-2,4,4-trimethylpentanesulphonic acid, 2-methacrylamidododecylsulphonic acid, 2-acrylamido-2,6-dimethyl-3-heptanesulphonic acid, and their partially or completely neutralized forms, and their mixtures.

More preferably, use is made of 2-acrylamido-2-methylpropanesulphonic acid (AMPS) and its partially or completely neutralized forms.

The amphiphilic polymers in accordance with the invention can preferably be selected from the group consisting of random amphiphilic AMPS polymers modified by reaction with a mono(C₆-C₂₂ n-alkyl)amine or a di(C₆-C₂₂ n-alkyl)amine, such as those disclosed in Patent Application WO-A-00/3 1154 (forming an integral, incorporated-by-reference part of the content of this description). These polymers can also comprise other ethylenically unsaturated hydrophilic monomers chosen, for example, from (meth)acrylic acids, their alkyl derivatives substituted at the β position or their esters obtained with monoalcohols or mono- or polyalkylene glycols, (meth)acrylamides, vinylpyrrolidone, maleic anhydride, itaconic acid, maleic acid, cationic monomers, such as diallyldimethylammonium chloride, or the mixtures of these compounds.

The preferred polymers of the invention are selected from the group consisting of polymers comprising polymerized units of amphiphilic copolymers of AMPS and of at least one hydrophobic monomer comprising ethylenic unsaturation comprising at least one hydrophobic part having from 6 to 50 carbon atoms, more preferably from 6 to 22 carbon atoms, more preferably still from 6 to 18 carbon atoms and more particularly 12 to 18 carbon atoms.

Aside from these monomers, these copolymers can additionally comprise polymerized units of one or more ethylenically unsaturated monomers not comprising a fatty chain (which is hydrophobic), such as (meth)acrylic acids, their alkyl derivatives substituted at the β position or their esters obtained with monoalcohols or mono- or polyalkylene glycols, (meth)acrylamides, vinylpyrrolidone, maleic anhydride, itaconic acid, maleic acid, cationic monomers, such as diallyl-dimethylammonium chloride, or the mixtures of these compounds.

These copolymers are disclosed in particular in the documents EP-A-750 899 and U.S. Pat. No. 5,089,578 and in the following publications by Yotaro Morishima:

“Self-assembling amphiphilic polyclectrolytes and their nanostructures”, Chinese Journal of Polymer Science, Vol. 18, No. 40 (2000), 323-336

“Micelle formation of random copolymers of sodium 2-(acrylamido)-2-methylpropanesulphonate and a non-ionic surfactant macromonomer in water as studied by fluorescence and dynamic light scattering”, Macromolecules, 2000, Vol. 33, No. 10, 3694-3704;

“Solution properties of micelle networks formed by non-ionic moieties covalently bound to a polyelectrolyte: salt effects on rheological behaviour”, Langmuir, 2000, Vol. 16, No. 12, 5324-5332;

Stimuli responsive amphiphilic copolymers of sodium 2-(acrylamido)-2-methylpropanesulfonate and associative macromonomers”, Polym. Preprint, Div. Polym. Chem., 1999, 40(2), 220-221, all incorporated herein by reference.

The hydrophobic monomers comprising ethylenic unsaturation of these specific copolymers are preferably selected from the group consisting of acrylates, alkylacrylates, acrylamides or alkylacrylamides of following formula (I):

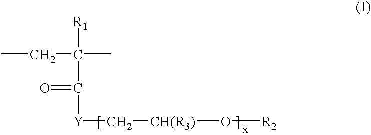

in which R₁ and R₃, which are identical or different, denote a hydrogen atom or a linear or branched C₁-C₆ alkyl radical (preferably methyl); Y denotes O or NH; R₂ denotes a hydrophobic hydrocarbonaceous radical comprising at least 6 carbon atoms, preferably from 6 to 50 carbon atoms, more preferably from 6 to 30 carbon atoms, more preferably still from 6 to 22 carbon atoms, more preferably still from 6 to 18 carbon atoms and more particularly from 12 to 18 carbon atoms; and x denotes a number of moles of alkylene oxide and varies from 0 to 100.

The R₂ radical is preferably selected from the group consisting of linear C₆-C 8 alkyl radicals (for example, n-hexyl, n-octyl, n-decyl, n-hexadecyl, n-dodecyl, n-octadecyl, n-behenyl or oleyl), branched C₆-C₁₈ alkyl radicals (for example, isostearyl) or cyclic C₆-C₁₈ alkyl radicals (for example, cyclododecyl (C₁₂) or adamantyl (C₁₀)); perfluorinated C₆-C₃₀ and preferably C₆-C₁₈ alkyl radicals (for example, the group of formula —(CH₂)₂—(CF₂)₉—CF₃); the cholesteryl radical (C₂₇) or a cholesterol ester residue, such as the cholesteryl oxyhexanoate group; polycyclic aromatic groups, such as naphthalene or pyrene; or silicone or alkylsilicone radicals or alkylfluorosilicone radicals. Preference is more particularly given, among these radicals, to linear alkyl radicals and more particularly the n-dodecyl, n-hexadecyl or n-octadecyl radical and their mixtures.

According to a particularly preferred form of the invention, the monomer of formula (I) comprises at least one alkylene oxide unit (x≧1) and preferably several alkylene oxide units (x>1), constituting a polyoxyalkylene chain. The polyoxyalkylene chain is preferably composed of ethylene oxide units and/or propylene oxide units and more particularly still is composed of ethylene oxide units. The number of oxyalkylene units (or number of alkylene oxide units) generally varies from 3 to 100, more preferably from 3 to 50 and more preferably still from 7 to 25.

Examples of these polymers include:

crosslinked or noncrosslinked and neutralized or unneutralized copolymers comprising from 15 to 60% by weight of AMPS units and from 40 to 85% by weight of (C₈-C₁₆)alkyl(meth)acrylamide units or of (C₈-C₁₆)alkyl (meth)acrylate units with respect to the polymer, such as those disclosed in Application EP-A-750 899;

terpolymers comprising from 10 to 90 mol % of acrylamide units, from 0.1 to 10 mol % of AMPS units and from 5 to 80 mol % of N-(C₆-C₁₈)alkylacrylamide units, such as those disclosed in U.S. Pat. No. 5,089,578.

Mention may also be made of crosslinked or noncrosslinked copolymers of partially or completely neutralized AMPS and of dodecyl, n-hexadecyl and/or n-octadecyl methacrylate, and crosslinked or noncrosslinked copolymers of AMPS and of n-dodecylmethacrylamide, such as those disclosed in the above-mentioned articles by Morishima.

Mention may more particularly be made of the copolymers composed of 2-acrylamido-2-methylpropanesulphonic acid (AMPS) units of following formula (II):

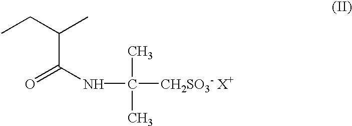

in which X⁺ is a proton, an alkali metal cation, an alkaline earth metal cation or the ammonium ion, and of units of following formula (III):

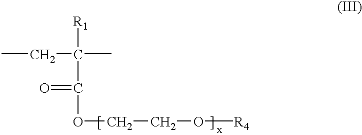

in which x denotes an integer varying from 3 to 100, preferably from 5 to 80 and more preferably from 7 to 25; R₁ has the same meaning as that indicated above in the formula (I) and R₄ denotes a linear or branched C₆-C₂₂ and more preferably C₁₀-C₂₂ alkyl.

The particularly preferred polymers are those for which x=25, R₁ denotes methyl (CH₃) and R₄ represents n-dodecyl; they are described in the abovementioned articles by Morishima. Other preferred polymers are those for which x=25, R₁ denotes methyl and R₄ represents n-hexadecyl (C₁₆) or n-octadecyl (C₁₈), or their mixtures.

Among noncrosslinked polymers, the particularly preferred polymers are obtained from AMPS units of formula (II) and from units of formula (III) where x=8, R₁ denotes the methyl radical (CH₃) and R₄=C₁₆₋₁₈.

The polymers for which X⁺ denotes sodium or ammonium are more particularly preferred.

The preferred amphiphilic polymers in accordance with the invention can be obtained according to conventional free-radical polymerization processes in the presence of one or more initiators, such as, for example, azobisisobutyronitrile (AlBN), azobisdimethylvaleronitrile, 2,2-azobis[2-amidinopropane] hydrochloride (ABAH), organic peroxides, such as dilauryl peroxide, benzyl peroxide, tert-butyl hydroperoxide and the like, inorganic peroxide compounds, such as potassium or ammonium persulphate, or H₂O₂, optionally in the presence of reducing agents.

The polymers can in particular be obtained by free-radical polymerization in a tert-butanol medium from which they precipitate. By using polymerization by precipitation from tert-butanol, it is possible to obtain a size distribution of the polymer particles which is particularly favourable for its uses.

The size distribution of the polymer particles can be determined, for example, by laser scattering or image analysis. An advantageous distribution for this type of polymer determined by image analysis is as follows: 60.2% less than 423 microns, 52.0% less than 212 microns, 26.6% less than 106 microns, 2.6% less than 45 microns and 26.6% greater than 850 microns.

The reaction can be carried out at a temperature of between 0 and 150° C., preferably between 10 and 1 00° C., either at atmospheric pressure or under reduced pressure. It can also be carried out under an inert atmosphere and preferably under nitrogen.

According to this process, 2-acrylamido-2-methylpropanesulphonic acid (AMPS) or one of its sodium or ammonium salts was in particular polymerized with an ester of (meth)acrylic acid and

of a C₁₀-C₁₈ alcohol oxyethylenated with 8 mol of ethylene oxide (Genapol® C-080 from Hoechst/Clariant),

of a C₁₁ oxo alcohol oxyethylenated with 8 mol of ethylene oxide (Genapol® UD-080 from Hoechst/Clariant),

of a C₁₁ oxo alcohol oxyethylenated with 7 mol of ethylene oxide (Genapol® UD-070 from Hoechst/Clariant),

of a C₁₂-C₁₄ alcohol oxyethylenated with 7 mol of ethylene oxide (Genapol® LA-070 from Hoechst/Clariant),

of a C₁₂-C₁₄ alcohol oxyethylenated with 9 mol of ethylene oxide (Genapol® LA-090 from Hoechst/Clariant),

of a C₁₂-C₁₄ alcohol oxyethylenated with 11 mol of ethylene oxide (Genapol® LA-110 from Hoechst/Clariant),

of a C₁₆-C₁₈ alcohol oxyethylenated with 8 mol of ethylene oxide (Genapol® T-080 from Hoechst/Clariant),

of a C₁₆-C₁₈ alcohol oxyethylenated with 15 mol of ethylene oxide (Genapol® T-150 from Hoechst/Clariant),

of a C₁₆-C₁₈ alcohol oxyethylenated with 11 mol of ethylene oxide (Genapol® T-110 from Hoechst/Clariant),

of a C₁₆-C₁₈ alcohol oxyethylenated with 20 mol of ethylene oxide (Genapol® T-200 from Hoechst/Clariant),

of a C₁₆-C₁₈ alcohol oxyethylenated with 25 mol of ethylene oxide (Genapol® T-250 from Hoechst/Clariant),

of a C₁₈-C₂₂ alcohol oxyethylenated with 25 mol of ethylene oxide and/or of a C₁₆-C₁₈ isoalcohol oxyethylenated with 25 mol of ethylene oxide.

The mol % concentration of the units of formula (II) and of the units of formula (III) in the polymers according to the invention varies according to the desired cosmetic application and the desired rheological properties of the formulation. For example, it can vary between 0.1 and 99.9 mol %.

Generally, for most hydrophobic polymers and in particular most hydrophobic crosslinked polymers, the molar proportion of units of formula (I) or (III) preferably varies from 50.1 to 99.9%, more particularly from 70 to 95% and more particularly still from 80 to 90%.

Generally, for the polymers which are not very hydrophobic and in particular the crosslinked polymers which are not very hydrophobic, the molar proportion of units of formula (I) or (III) preferably varies from 0.1 to 50%, more particularly from 5 to 25% and more particularly still from 10 to 20%.

For the most hydrophobic noncrosslinked polymers, the molar proportion of units of formula (I) or (III) preferably varies from 40 to 99.9%, more particularly from 50 to 90% and more particularly still from 60 to 80%. For the noncrosslinked polymers which are not very hydrophobic, the molar proportion of units of formula (I) or (III) preferably varies from 0.1 to 50%, more particularly from 1 to 25% and more particularly still from 2 to 20%.

According to a preferred embodiment of the invention, the amphiphilic polymer used as noncrosslinked polymer is a copolymer of AMPS and of C₁₆-C₁₈ alkyl methacrylate comprising 8 oxyethylene groups, obtained from AMPS or from an AMPS salt and from Genapol T-080 or from Genapol C-080.

The distribution of the monomers in the polymers of the invention can, for example, be alternating, block (including multiblock) or random.

According to another embodiment of the invention, the polymers can have heat-sensitive pendent chains and exhibit an aqueous solution having a viscosity which, beyond a certain threshold temperature, increases or remains virtually constant when the temperature increases. It then relates more particularly to polymers, the aqueous solution of which exhibits a viscosity which is low below a first threshold temperature and which, above this first threshold temperature, increases towards a maximum when the temperature increases and which, above a second threshold temperature, again decreases when the temperature increases. From this viewpoint, it is preferable for the viscosity of the polymer solutions below the first threshold temperature to be from 5 to 50%, in particular from 10 to 30%, of the maximum viscosity at the second threshold temperature. These polymers preferably result, in water, in a phenomenon of phase separation by heating, reflected by curves exhibiting, as a function of the temperature and of the concentration, a minimum known as the LCST (Lower Critical Solution Temperature). The viscosities (measured at 25° C. with a Brookfield viscometer, needle 7) of the 1% aqueous solutions of such polymers preferably range from 20 000 mPa.s to 100 000 mPa.s and more particularly from 60 000 mPa.s to 70 000 mPa.s.

The amphiphilic polymers according to the invention are preferably present in the compositions in amounts ranging from 0.01 to 20% by weight, more preferably from 0.1 to 10%, more preferably still from 0.1 to 5% by weight and more particularly still from 0.5 to 2% by weight with respect to the total weight of the composition. More particularly, the noncrosslinked amphiphilic polymers used in accordance with the invention are preferably present in the composition of the invention in an amount equal to or less than 1% by weight with respect to the total weight of the composition. They can be present, for example, in amounts ranging from 0.01 to 1% by weight of active material, more preferably from 0.05 to 0.7% of active material, more preferably still from 0.1 to 0.6% by weight of active material, with respect to the total weight of the emulsion.

Preferably, the amphiphilic polymer is introduced into the aqueous phase of the emulsion according to the invention.

The composition according to the invention preferably comprises an amount of aqueous phase ranging from 50 to 95% by weight and preferably from 60 to 80% by weight with respect to the total weight of the composition. In the particular case of the composition according to the invention comprising at least 40% of oily phase, the amount of aqueous phase generally ranges from 20 to 60% by weight, preferably from 25 to 55% by weight and better still from 30 to 50% by weight with respect to the total weight of the composition.

The aqueous phase comprises water and any other water-soluble compound optionally present, such as, in particular, water-soluble solvents and additives (for example, surfactants and active principles).

Mention may in particular be made, as useful water-soluble solvents, of lower alcohols and of polyols. The term “lower alcohols” is understood to mean alcohols comprising from 1 to 8 and more particularly from 1 to 6 carbon atoms, such as ethanol. Mention may be made, as polyols, of, for example, glycerol, propylene glycol, butylene glycol and polyethylene glycols (PEG-8 for example). These alcohols and/or polyols can be present in the composition in any amount, preferably an amount ranging from 0.1 to 25% and from 1 to 15% of the total weight of the composition.

The oily phase of the composition according to the invention may comprise oils and other fatty substances, in addition to fat-soluble additives (for example, surfactants and active principles). It comprises at least one make-up-removing oil preferably selected from the group consisting of hydrocarbonaceous oils, fatty esters comprising at least 8 carbon atoms, and their mixtures.

The term “hydrocarbonaceous oil” is understood here to mean any oil predominantly comprising carbon and hydrogen atoms. Mention may be made, as hydrocarbonaceous oils, of, for example, liquid paraffins, which may or may not be volatile, and their derivatives, liquid petrolatum, polydecenes, hydrogenated polyisobutene, such as Parleam® oil, hydrogenated isoparaffin, isohexadecane, isododecane, and their mixtures.

The term “fatty ester” is understood to mean an ester obtained from a fatty acid and/or from a fatty alcohol. Fatty esters comprise at least 8 carbon atoms and preferably from 9 to 26 carbon atoms and better still from 12 to 22 carbon atoms.

The ester constituting the make-up-removing oil can preferably be chosen in from:

(1) the esters obtained from a fatty acid with a straight or branched chain comprising at least 9 carbon atoms and from alcohols with a straight or branched chain comprising from 1 to 17 carbon atoms;

(2) the esters obtained from an acid with a straight or branched chain comprising 1 to 17 carbon atoms and from a fatty alcohol with a straight or branched chain comprising at least 9 carbon atoms;

(3) the esters obtained from benzoic acid and from a fatty alcohol with a straight or branched chain comprising at least 9 carbon atoms; and

(4) their mixtures.

The esters are preferably mono- or diesters.

The ester which can constitute the make-up-removing oil is preferably selected from the group consisting of the group of esters comprising no unsaturation and/or no ether or hydroxyl group. More advantageously still, it is a saturated ester which includes neither an ether group nor a hydroxyl group.

Thus, the ester which can be used as make-up-removing oil of the composition in accordance with the invention can be chosen in particular from the group consisting of 2-ethylhexyl palmitate (or octyl palmitate), isononyl isononanoate, isostearyl lactate, 2-ethylhexyl myristate (or octyl myristate), isopropyl palmitate, isopropyl myristate, diisopropyl adipate, dioctyl adipate, 2-ethylhexyl hexanoate, ethyl laurate, methyl myristate, octyldodecyl octanoate, isodecyl neopentanoate, ethyl myristate, myristyl propionate, 2-ethylhexyl 2-ethylhexanoate, 2-ethylhexyl octanoate, 2-ethylhexyl caprate/caprylate, methyl palmitate, butyl myristate, isobutyl myristate, ethyl palmitate, isohexyl laurate, hexyl laurate, isopropyl isostearate, the ester of benzoic acid and of C₁₂-C₁₅ alcohols (CTFA name: C12-15 Alkyl Benzoate), and their mixtures.

The amount of make-up-removing oil(s) can constitute all or a portion of the oily phase and, for example, from 1 to 100%, preferably 1 to 90%, more preferably 5 to 80%, better still 5 to 70% and even better still 10 to 70%, of the total weight of the oily phase.

The oily phase can additionally comprise any fatty substance and in particular non-make-up-removing oil, other than those indicated above, conventionally used in the cosmetics field. Mention may be made, as other oils capable of being present in the oily phase, of, for example, oils of vegetable origin, such as apricot kernel oil; synthetic oils; volatile or nonvolatile silicone oils; and fluorinated oils. The other fatty substances capable of being present in the oily phase can, for example, be fatty acids, fatty alcohols and waxes. Mention may be made, as silicone oils, of, for example, volatile or nonvolatile polymethylsiloxanes (PDMS) comprising a linear or cyclic silicone chain which are liquid or pasty at ambient temperature, in particular cyclopolydimethylsiloxanes (cyclomethicones), such as cyclohexasiloxane; polydimethylsiloxanes comprising pendent alkyl, alkoxy or phenyl groups or alkyl, alkoxy or phenyl groups at the end of the silicone chain, which groups have from 2 to 24 carbon atoms; phenylated silicones, such as phenyl trimethicones, phenyl dimethicones, phenyltrimethylsiloxydiphenylsiloxanes, diphenyl dimethicones, diphenylmethyldiphenyltrisiloxanes, (2-phenylethyl)trimethylsiloxysilicates and polymethylphenylsiloxanes; their mixtures.

According to a preferred embodiment of the invention, the composition of the invention comprises at least one make-up-removing oil as defined above and at least one oil selected from the group consisting of silicone oils. More preferably, the composition comprises at least one ester and/or at least one hydrocarbonaceous oil and at least one silicone oil.

The oily phase is present in the composition according to the invention in an amount ranging from 15 to 60% by weight and preferably from 20 to 40% by weight with respect to the total weight of the composition. In the case of a composition comprising at least 40% of oily phase, the oily phase is present in an amount which can range, for example, from 40 to 80% by weight, preferably from 45 to 75% by weight and better still from 50 to 70% by weight with respect to the total weight of the composition.

The composition of the invention can additionally comprise one or more adjuvants selected from the group consisting of the adjuvants useful in the cosmetics field, such as hydrophilic or lipophilic active principles, preservatives, antioxidants, fragrances, solvents, fillers, fibres, sunscreen agents, colouring materials (pigments or pearlescence agents), pH adjusters (bases or acids, such as citric acid), lipid vesicles, polymers other than those indicated above, and their mixtures. These adjuvants are used in the proportions usual in the cosmetics or dermatological field, for example from 0.01 to 30% of the total weight of the composition, and they are, depending on their nature, introduced into the aqueous phase or into the oily phase of the emulsion or into vesicles. These adjuvants and their concentrations must be such that they do not modify the property desired for the composition.

Thus, it is possible to incorporate, in the composition of the invention, one or more polymers in concentrations preferably ranging from 0.05 to 2% by weight with respect to the total weight of the composition. Mention may be made, as examples of polymers which can be used in the composition of the invention, of:

polysaccharide biopolymers, such as xanthan gum, guar gum, alginates or modified celluloses;

synthetic polymers, such as polyacrylics, for example Carbopol 980, sold by Goodrich, or acrylate/acrylonitrile copolymers, such as Hypan SS201, sold by Kingston;

inorganic compounds, such as smectites or modified or unmodified hectorites, such as the Bentone products sold by Rheox, the Laponite products sold by Southern Clay Products or the Veegum HS product sold by R. T. Vanderbilt;

and their mixtures.

Mention may in particular be made, as active principles which can be used in the composition of the invention, of antiseborrhoeic and antimicrobial active principles which make possible in particular the treatment of greasy skin. This active principle can be chosen in particular from β-lactam derivatives, quinolone derivatives, ciprofloxacin, norfloxacin, tetracycline and its salts, erythromycin and its salts, amikacin and its salts, 2,4,4′-trichloro-2′-hydroxydiphenyl ether (or triclosan), 3,4,4′-trichlorocarbanilide (or triclocarban), phenoxyethanol, phenoxypropanol, phenoxyisopropanol, doxycycline and its salts, capreomycin and its salts, chlorhexidine and its salts, chlortetracycline and its salts, oxytetracycline and its salts, clindamycin and its salts, ethambutol and its salts, hexamidine isethionate, metronidazole and its salts, pentamidine and its salts, gentamicin and its salts, kanamycin and its salts, lineomycin and its salts, methacycline and its salts, methenamine and its salts, minocycline and its salts, neomycin and its salts, netilmicin and its salts, paromomycin and its salts, streptomycin and its salts, tobramycin and its salts, miconazole and its salts, amanfadine salts, para-chloro-meta-xylenol, nystatin, tolnaftate, salicylic acid and its salts, 5-(n-octanoyl)salicylic acid and its salts, benzoyl peroxide, 3-hydroxybenzoic acid, 4-hydroxybenzoic acid, acetylsalicylic acid, 2-hydroxybutanoic acid, 2-hydroxypentanoic acid, 2-hydroxyhexanoic acid, phytic acid, N-acetyl-L-cysteine, lipoic acid, azelaic acid, arachidonic acid, ibuprofen, naproxen, hydrocortisone, acetaminophen, resorcinol, octopirox, lidocaine hydrochloride, clotrimazole, octoxyglycerin, octanoylglycine, caprylylglycol, 10-hydroxy-2-decanoic acid, zinc salts, such as zinc gluconate, niacinamide or vitamin B3 (or vitamin PP), and their mixtures.

Mention may also be made, as active principles which can be used in the composition of the invention, of keratolytic and anti-ageing agents, such as hydroxy acids (α-hydroxy acids and β-hydroxy acids), for example lactic acid, glycolic acid, citric acid, salicylic acid and their derivatives; essential oils; vitamins and in particular retinol (vitamin A), ascorbic acid (vitamin C), tocopherol (vitamin E), niacinamide (vitamin PP or B3), panthenol (vitamin B5) and their derivatives (for example, esters); coenzymes and in particular coenzyme Q10 or ubiquinone; enzymes, such as, for example, lipases, proteases, phospholipases, cellulases, peroxidases, in particular lactoperoxidases, catalases, superoxide dismutases and the plant extracts comprising the abovementioned enzymes; yeasts, such as Saccharomyces cerevisiae; steroids; antioxidants and agents for combating free radicals; moisturizing agents, such as polyols (glycerol, sorbitol or sugars), protein hydrolysates, urea and the mixtures comprising them; slimming active principles, such as caffeine; antielastase and anticollagenase agents; agents for the treatment of greasy skin; plant extracts and in particular plankton extracts; tightening agents; mattifying agents; antiperspirants, such as aluminium salts; and their mixtures.

Mention may in particular be made, as salicylic acid derivatives, of 5-(n-octanoyl)salicylic acid (CTFA name: Capryloyl Salicylic Acid), 5-(n-decanoyl)salicylic acid, 5-(n-dodecanoyl)salicylic acid, 5-(n-octyl)salicylic acid, 5-(n-heptyloxy)salicylic acid, 5-(tert-octyl)salicylic acid, 5-butoxysalicylic acid, 5-ethoxysalicylic acid, 5-methoxysalicylic acid, 5-propoxysalicylic acid, 5-methylsalicylic acid, 5-ethylsalicylic acid, 5-propylsalicylic acid, and their mixtures. These acids can optionally be salified with a base.

Mention may be made, as examples of steroids, of, for example, dehydroepiandrosterone (or DHEA), and (1) its biological derivatives and precursors, in particular DHEA salts and esters, such as DHEA sulphate and salicylate, 7-hydroxy-DHEA, 7-keto-DHEA, or 7-hydroxy- and 7-keto-DHEA esters, in particular 3-β-acetoxy-7-oxo-DHEA, and (2) its chemical derivatives and precursors, in particular sapogenins, such as diosgenin or hecogenin, and/or their derivatives, such as hecogenin acetate, and/or the natural extracts comprising them and in particular extracts of Dioscorea species, such as wild yam.

According to a specific embodiment of the invention, the active principle is selected from the group consisting of hydroxy acids, vitamins, plankton extracts and their mixtures.

The fibres which can be used in the composition of the invention can be hydrophilic or hydrophobic fibres of synthetic or natural and inorganic or organic origin. The fibres can, for example, be those used in the manufacture of textiles and in particular fibres of silk, cotton, wool, flax, cellulose, which fibres are extracted in particular from wood, plants or algae, polyamide (Nylon®), modified cellulose (rayon, viscose or acetate, in particular rayon acetate), poly(p-phenylene terephthalamide), in particular Kevlar®, acrylic, in particular poly(methyl methacrylate) or poly(2-hydroxyethyl methacrylate), polyolefin and in particular polyethylene or polypropylene, glass, silica, aramide, carbon, in particular in the graphite form, Teflon®, insoluble collagen, polyesters, poly(vinyl chloride), poly(vinylidene chloride), poly(vinyl alcohol), polyacrylonitrile, chitosan, polyurethane or poly(ethylene phthalate), fibres formed from a blend of polymers such as those mentioned above, for example polyamide/polyester fibres, and their mixtures. Use may also be made of resorbable synthetic fibres used in surgery, such as the fibres prepared from glycolic acid and caprolactone (Monocryl from Johnson & Johnson); resorbable synthetic fibres of the copolymer of lactic acid and of glycolic acid type (Vicryl from Johnson & Johnson); terephthalic polyester fibres (Ethibond from Johnson & Johnson); and stainless steel wires (Acier from Johnson & Johnson). Use may also be made of a mixture of these various fibres. These fibres can be present in amounts ranging from 0 to 20% by weight and preferably from 0.5 to 10% by weight with respect to the total weight of the composition.

The composition of the invention can additionally comprise fillers, which can be used for the purpose of modifying the texture of the composition. Mention may be made, as fillers which can be used in the composition of the invention, of, for example, in addition to pigments, silica powder; talc; polyamide particles and in particular those sold under the name Orgasol by Atochem; polyethylene powders; microspheres based on acrylic copolymers, such as those made of ethylene glycol dimethacrylate/lauryl methacrylate copolymer which are sold by Dow Corning under the name of Polytrap; expanded powders, such as hollow microspheres and in particular the microspheres sold under the name Expancel by Kemanord Plast or under the name Micropearl F 80 ED by Matsumoto; powders formed of natural organic materials, such as maize, wheat or rice starches, which may or may not be crosslinked, such as powders formed of starch crosslinked with octenylsuccinic anhydride which are sold under the name Dry-Flo by National Starch; silicone resin microbeads, such as those sold under the name Tospearl by Toshiba Silicone; and their mixtures. These fillers can be present in amounts ranging from 0 to 40% by weight and preferably from 1 to 10% by weight with respect to the total weight of the composition.

The sunscreen agents (or UV screening agents) optionally present in the composition are not limited and can be selected from the group consisting of organic screening agents, physical screening agents, such as titanium oxide or zinc oxide, and their mixtures.

The more particularly preferred organic UV screening agents are selected from the group consisting of the following compounds:

Salicylic derivatives and in particular ethylhexyl salicylate, sold under the trade name Neo Heliopan OS by Haarmann and Reimer;

Dibenzoylmethane derivatives and in particular butyl methoxydibenzoylmethane, sold in particular under the trade name Parsol 1789 by Hoffmann-LaRoche;

Cinnamic derivatives and in particular ethylhexyl methoxycinnamate, sold in particular under the trade name Parsol MCX by Hoffmann-LaRoche;

β,β-Diphenylacrylate derivatives and in particular octocrylene (2-ethylhexyl α-cyano-β,β-diphenylacrylate), sold in particular under the trade name Uvinul N539 by BASF;

Phenylbenzimidazole sulphonic acid;

Benzylidene camphor derivatives and in particular terephthalylidene dicamphor sulfonic acid, manufactured under the name Mexoryl SX by Chimex, and 4

methylbenzylidene camphor, sold under the trade name Eusolex 6300 by Merck;

Benzophenone derivatives and in particular benzophenone-3 or oxybenzone, sold under the trade name Uvinul M40 by BASF; benzophenone-4, sold under the trade name Uvinul MS40 by BASF; or benzophenone-5;

Phenylbenzimidazole derivatives and in particular benzimidazilate, sold under the trade name Neo Heliopan AP by Haarmann and Reimer;

Triazine derivatives and in particular anisotriazine, sold under the trade name Tinosorb S by Ciba Geigy; ethylhexyl triazone, sold in particular under the trade name Uvinul T150 by BASF; and diethylhexyl butamido triazone, sold under the trade name Uvasorb HEB by Sigma 3V;

Methylene bis-benzotriazolyl tetramethylbutylphenol;

Phenylbenzotriazole derivatives and in particular drometrizole trisiloxane, sold under the trade name Silatrizole by Rhodia Chimie;

and their mixtures.

Mention may be made, as physical screening agents which can be added to the composition of the invention, of, for example, coated or uncoated metal oxide pigments and nanopigments, in particular titanium, iron, zirconium, zinc or cerium oxides, and their mixtures, it being possible for these oxides to be in the form of microparticles or nanoparticles (nanopigments) which are optionally coated.

The person skilled in the art is able to choose the optional compound or compounds to be added to the composition according to the invention so that the advantageous properties intrinsically attached to the composition in accordance with the invention are not, or not substantially, detrimentally affected by the envisaged addition.

The compositions according to the invention can be more or less fluid and they can therefore be provided in the form of a lotion, of a milk or in the form of a more or less thick cream. They can in particular constitute products for removing make-up from and/or cleaning the skin, lips and/or eyes.

A further subject-matter of the present invention is a cosmetic process for removing make-up from and/or cleaning the skin, lips and/or eyes, wherein a composition as defined above is applied to the skin, lips and/or eyes.

Another subject-matter of the invention is the cosmetic use of the composition as defined above for removing make-up from and/or cleaning the skin, lips and/or eyes.

The example below of compositions according to the invention is given by way of illustration and without a limiting nature. The amounts are given therein as % by weight, unless otherwise mentioned.

### EXAMPLE 1 (ACCORDING TO THE INVENTION)

### O/W Emulsion

| A. Oily phase | | |
|---|---|---|
| Cyclopentadimethylsiloxane | | 8% |
| Isododecane | | 6% |
| Isononyl isononanoate | | 6% |
| B. Aqueous phase | | |
| Crosslinked AMPS copolymer (*) | | 1% |
| Preservatives | | q.s. |
| Water | q.s. for | 100% |

(*) Copolymer crosslinked with allyl methacrylate, composed of 80% by weight of AMPS units neutralized with NH3 and of 20% by weight of Genapol T-250 methacrylate units [units of formula (III) in which R₁=CH₃, R₄=C₁₆-C₁₈ and x=25]. Procedure: The copolymer is dispersed in the aqueous phase at 80° C. with stirring and then the oily phase is dispersed with stirring in the aqueous gel obtained.

A gelled, soft, homogeneous, smooth and glossy white cream is obtained. Under the microscope, it has an even appearance with sharp edges. This cream exhibits excellent stability after 2 months at 45° C.

### EXAMPLE 2 (COMPARATIVE EXAMPLE)

### O/W Emulsion

| A. Oily phase | | |
|---|---|---|
| Cyclopentadimethylsiloxane | | 8% |
| Isododecane | | 6% |
| Isononyl isononanoate | | 6% |
| B. Aqueous phase | | |
| AMPS polymer (Hostacerin AMPS from Clariant) | | 1% |
| Preservatives | | q.s. |
| Water | q.s. for | 100% |

Procedure: The polymer is dispersed in the aqueous phase at 80° C. with stirring and then the oily phase is dispersed with stirring in the aqueous gel obtained.

A gelled, soft, homogeneous, smooth and glossy white cream is obtained. However, under the microscope, the emulsion has an uneven appearance with edges which are not always sharp. After 2 months at 45° C., it is seen, under the microscope, that the oil globules are large and that there are oil “failures”, that is to say that, at certain points, the oil globules have joined together and have formed a large blob.

### EXAMPLE 3 (COMPARATIVE)

### O/W Emulsion

| A. Oily phase | | |
|---|---|---|
| Cyclopentadimethylsiloxane | | 8% |
| Isododecane | | 6% |
| Isononyl isononanoate | | 6% |
| PEG-20 stearate | | 0.25% |
| B. Aqueous phase | | |
| AMPS homopolymer (Hostacerin AMPS) | | 1% |
| Preservatives | | q.s. |
| Water | q.s. for | 100% |

Procedure: The homopolymer is dispersed in the aqueous phase at 80° C. with stirring, then the oily phase is prepared and the oily phase is dispersed with stirring in the aqueous gel obtained.

A gelled, soft, homogeneous, smooth and glossy white cream is obtained. Under the microscope, it has an even appearance with sharp edges. This cream exhibits excellent stability after 2 months at 45° C.

The stability and the make-up-removing effectiveness of the three formulas were evaluated:

Stability:

| | | Example 2 | Example 3 |
|---|---|---|---|
| Test | Example 1 | (comparative) | (comparative) |
| Polymers | Copolymer: | Homopolymer: | Homopolymer: |
| | 1% | 1% | 1% |
| PEG stearate | — | — | 0.25% |
| Oily phase: | | | |
| Cyclopentadi- | 8% | 8% | 8% |
| methylsiloxane | | | |
| Isododecane | 6% | 6% | 6% |
| Isononyl | 6% | 6% | 6% |
| isononanoate | | | |
| Appearance under | Nothing to | Large globules and | Nothing to |
| the microscope after | report | failures | report |
| 2 months at 45° C. | | | |

Make-Up-Removing Power:

The make-up-removing power of the examples indicated above was tested using transfer-free lipstick to confirm the effectiveness of the make-up removal.

The test is carried out in the following way:

1) The transfer-free lipstick is applied to the forearm in two to and fro movements over a range of three centimetres. The surface greasy part is allowed to dry for 5 minutes;

2) An identical amount (i.e. three doses with a pump-action spray) of each of the compositions is respectively deposited on the lipstick marks;

3) The cream is spread over each lipstick mark by massaging with a finger in ten circular movements;

4) The marks are wiped off with a make-up-removing cotton pad in one movement from the top downwards.

An excellent make-up-removing power is observed with the compositions of Examples 1 and 2 and a much poorer make-up-removing power with the composition of Example 3. However, as the composition of Example 2 is less stable, only the composition of the invention exhibits both an excellent make-up-removing power and good stability. Furthermore, this test shows, surprisingly, that the addition of surfactant reduces the make-up-removing power of the composition.

### EXAMPLES 5 TO 8 ACCORDING TO THE INVENTION

The process for the preparation of the compositions of Examples 5 to 8 is as follows: Al is dispersed in water with A4 with stirring and then the other constituents of phase A are added; B is emulsified in A at 25° C. with mechanical stirring until a smooth and glossy white emulsion is formed.

| Examples | O/W emulsion | No.5 | No.6 | No.7 | No.8 |
|---|---|---|---|---|---|
| | | 60% of base B | | 40% of phase B | |
| Aqueous | A1-AMPS/C₁₆₋₁₈alkyl | 0.4 | 0.4 | 0.4 | 0.6 |
| phase A | methacrylate(8 EO) | | | | |
| | copolymer | | | | |
| | A2-water | q.s for 100 | q.s for 100 | q.s for 100 | q.s for 100 |
| | A3-glycerol | 3 | 3 | 3 | 3 |
| | A4-base (sodium | 0.07 | 0.07 | 0.07 | 0.0105 |
| | A5—preservative | 0.1 | 0.1 | 0.1 | 0.1 |
| Oily | Cyclopentasiloxane | 12 | 36 | — | — |
| phase B | Liquid petrolatum | 31.8 | | | |
| | Isopropyl palmitate | 16.2 | | 24 | 24 |
| | Isohexadecane | — | 24 | | |
| | Isoclodecane | — | | 36 | 36 |

| | | | | |
|---|---|---|---|---|
| Monitoring operations at 24 hours | pH = 7.23 | pH = 6.91 | pH = 7.43 | pH = 7 |
| | viscosity = | viscosity = | viscosity = | viscosity = |
| | 19 poises | 464 cPoises | 15 poises | 4.6 poises |
| | (=1.9 Pa · s) | (=0.464 Pa · s) | (= 1.5 Pa · s) | (=0.46 Pa · s |
| | size of the | size of the | size of the | |
| | oily globules = | oily globules = | oily globules = | |
| | 7 μm | 20 μm | 28 μm | |
| | Fine, homo-geneous and | Mote fluid, | Fine and homogeneous | Emulsion with a |
| | glossy white emulsion | homogeneous, white | emulsion | white and homo- |
| | | emulsion | | geneous appearance |
| Stability after spending 2 months | Stable | Stable | Stable | Stable |
| at 4° C., 25° C. and 45° C. | | | | |

These emulsions exhibit the following advantages:

They are therefore stable after spending 2 months at different temperatures (4° C., 25° C., 45° C.).

They can have a fairly high viscosity (approximately 15 poises, i.e. 1.5 Pa.s, on a Rheomat 180 at 25° C., 200 s⁻¹). In that case, the composition is not a milk but a soft cream and it exhibits the advantage of not flowing on application to the skin (Examples No. 5 and 7).

Oils of several natures are compatible, both silicones and alkanes or esters or others.

These emulsions are stable without addition of other surface-active agents (emulsifying surfactant, cosurfactant, such as fatty alcohols).

Make-Up-Removing Power:

The make-up-removing power of Examples 5 to 8 indicated above was tested using a transfer-free foundation to confirm the effectiveness of the make-up removal.

The test was carried out in the following way:

5) The transfer-free foundation, “Air Wear” from L'Oreal Paris, is applied in an amount of 50 mg to a defined area of 4×4 cm of the forearm;

6) At the same time, the make-up-removing formulas are weighed (100 mg) and deposited on a cotton pad;

7) After drying the make-up (approximately 15 minutes), the cotton pad, impregnated with make-up remover, is applied via 5 movements (from the top downwards) to the defined made-up region;

8) Subsequently, the make-up removal obtained is compared with respect to the bare skin.

In this test, the formulas Nos. 5, 6 and 7 showed excellent make-up removal and the formula No. 8 showed reasonable make-up removal.

The effectiveness of the make-up removal, the comfort and the sensory evaluation of the formula No. 7 were also tested on a panel of 27 women who used it for 10 days. It emerged from this test that the product had a make-up-removing power judged to be good, very good cosmetic aspects, easy rinsing and good comfort.

All references, texts, patents, applications, standards, etc mentioned herein are incorporated by reference. Where a numerical range is stated all values and subranges therebetween are included as if specifically written out.

The above description and disclosure makes able the ordinary worker to make and use the invention as described herein and as set out in the claims that follow.

1. A cosmetic composition comprising an oily phase dispersed in an aqueous phase, and
(1) an amphiphilic polymer comprising at least one polymerized monomer unit comprising ethylenic unsaturation comprising a sulphonic group, in the free or partially or completely neutralized form, and comprising at least one hydrophobic part, and
(2) at least one make-up-removing oil.

2. The composition according to claim 1, wherein the hydrophobic part of the amphiphilic polymer comprises from 6 to 30 carbon atoms.

3. The composition according to claim 1, wherein the hydrophobic part of the amphiphilic polymer comprises from 6 to 50 carbon atoms.

4. The composition according to claim 1, wherein the hydrophobic part of the amphiphilic polymer comprises from 6 to 22 carbon atoms.

5. The composition according to claim 1, wherein the amphiphilic polymer is partially or completely neutralized by an inorganic or organic base.

6. The composition according to claim 1, wherein the amphiphilic polymer has a number-average molecular weight ranging from 1 000 to 20 000 000 g/mol.

7. The composition according to claim 1, wherein a 1% by weight aqueous solution of the said polymer exhibits, at a temperature of 25° C., a viscosity, measured with a Brookfield viscometer, needle 7, ranging from 20 000 mPa.s to 100 000 mPa.s.

8. The composition according to claim 1, wherein the amphiphilic polymer is prepared by free-radical polymerization by precipitation from tert-butanol.

9. The composition according to claim 1, wherein the amphiphilic polymer is crosslinked.

10. The composition according to claim 9, wherein the polymer is crosslinked with a crosslinking agent or agents selected from the group consisting of compounds comprising olefinic polyunsaturation.

11. The composition according to claim 1, wherein the crosslinking agent or agents are selected from the group consisting of methylenebisacrylamide, allyl methacrylate and trimethylolpropane triacrylate (TMPTA).

12. The composition according to claim 10, wherein the degree of crosslinking varies from 0.01 to 10 mol % with respect to the polymer.

13. The composition according to claim 1, wherein the monomer comprising ethylenic unsaturation comprising a sulphonic group is selected from the group consisting of vinylsulphonic acid, styrenesulphonic acid, (meth)acrylamido(C₁-C₂₂)alkylsulphonic acids, N-(C₁-C₂₂)alkyl(meth)acrylamido(C₁-C₂₂)alkylsulphonic acids, and their partially or completely neutralized forms, and their mixtures.

14. The composition according to claim 1, wherein the monomer comprising ethylenic unsaturation comprising a sulphonic group is selected from the group consisting of acrylamidomethanesulphonic acid, acrylamidoethanesulphonic acid, acrylamidopropanesulphonic acid, 2-acrylamido-2-methylpropanesulphonic acid, 2-methacrylamido-2-methylpropanesulphonic acid, 2-acrylamido-n-butanesulphonic acid, 2-acrylamido-2,4,4-trimethylpentanesulphonic acid, 2-methacrylamidododecylsulphonic acid, 2-acrylamido-2,6-dimethyl-3-heptanesulphonic acid, and their partially or completely neutralized forms, and their mixtures.

15. The composition according to claim 13, wherein the monomer comprising ethylenic unsaturation comprising a sulphonic group is selected from the group consisting of 2-acrylamido-2-methylpropanesulphonic acid (AMPS), and its partially or completely neutralized forms.

16. The composition according to claim 1, wherein the amphiphilic polymer is selected from the group consisting of random AMPS polymers modified by reaction with a mono(C₆-C₂₂ n-alkyl)amine or a di(C₆-C₂₂ n-alkyl)amine.

17. The composition according to claim 15, wherein the amphiphilic AMPS polymer additionally comprises at least one polymerized monomer comprising ethylenic unsaturation not comprising a fatty chain.

18. The composition according to claim 1, wherein the monomer comprising ethylenic unsaturation not comprising a fatty chain is selected from the group consisting of (meth)acrylic acids and their alkyl derivatives substituted at the P position and their esters obtained with monoalcohols or mono- or polyalkylene glycols, or else from (meth)acrylamides, vinylpyrrolidone, maleic anhydride, itaconic acid, maleic acid or cationic monomers, or the mixtures of these compounds.

19. The composition according to claim 1, wherein the amphiphilic polymer is selected from the group consisting of amphiphilic copolymers of AMPS and of at least one hydrophobic monomer comprising ethylenic unsaturation comprising at least one hydrophobic part having from 6 to 50 carbon atoms.

20. The composition according to claim 1, wherein the hydrophobic part is selected from the group consisting of acrylates, alkylacrylates, acrylamides or alkylacrylamides of following formula (I): 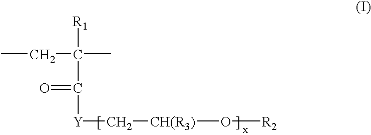
in which R₁ and R₃, which are identical or different, denote a hydrogen atom or a linear or branched C₁-C₆ alkyl radical; Y denotes O or NH; R₂ denotes a hydrophobic hydrocarbonaceous radical comprising from 6 to 50 carbon atoms; and x denotes a number of moles of alkylene oxide and varies from 0 to 100.

21. The composition according to claim 1, wherein the hydrophobic radical R₂ is selected from the group consisting of linear, branched or cyclic C₆-C₁₈ alkyl radicals; perfluorinated C₆-C₁₈ alkyl radicals; the cholesteryl radical or a cholesterol ester residue; or polycyclic aromatic groups.

22. The composition according to claim 20, wherein the monomer of formula (I) comprises at least one alkylene oxide unit (x≧1).

23. The composition according to claim 20, wherein the number of alkylene oxide units varies from 3 to 100.

24. The composition according to claim 20, wherein the number of alkylene oxide units varies from 3 to 50.

25. The composition according to claim 20, wherein the portion of the monomer of formula (I) denoted by X comprises solely ethylene oxide units.

26. The composition according to claim 19, wherein the amphiphilic AMPS polymer is selected from the group consisting of:
crosslinked or noncrosslinked and neutralized or unneutralized copolymers comprising from 15 to 60% by weight of AMPS units and from 40 to 85% by weight of (C₈-C₁₆)alkyl(meth)acrylamide units or of (C₈-C₁₆)alkyl (meth)acrylate units with respect to the polymer;
terpolymers comprising from 10 to 90 mol % of acrylamide units, from 0.1 to 10 mol % of AMPS units and from 5 to 80 mol % of N-(C₆-C₁₈)alkylacrylamide units with respect to the polymer;
crosslinked or noncrosslinked copolymers of partially or completely neutralized AMPS and of n-dodecyl, n-hexadecyl and/or n-octadecyl methacrylate;
crosslinked or noncrosslinked copolymers of partially or completely neutralized AMPS and of n-dodecylmethacrylamide.

27. The composition according to claim 19, wherein the amphiphilic AMPS polymer is selected from the group consisting of the copolymers composed of 2-acrylamido-2-methylpropanesulphonic acid (AMPS) units of following formula (II): 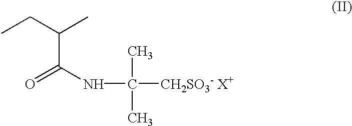
in which X⁺ is a proton, an alkali metal cation, an alkaline earth metal cation or the ammonium ion,
and of units of following formula (III): 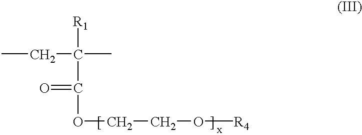
in which x denotes an integer varying from 3 to 100; R₁ has the same meaning as that indicated above in the formula (I) and R₄ denotes a linear or branched C₆-C₂₂ alkyl.

28. The composition according to claim 27, wherein x=25, R₁ is methyl and R₄ is n-dodecyl, n-hexadecyl or n-octadecyl.

29. The composition according to claim 27, wherein x=8, R₁ denotes the methyl radical and R₄ is C_{16-18.}

30. The composition according to claim 1, wherein the amount of amphiphilic polymer(s) varies from 0.01 to 20% by weight with respect to the total weight of the composition.

31. The composition according to claim 1, wherein the amount of oily phase ranges from 15 to 60% by weight of the total weight of the composition.

32. The composition according to claim 1, wherein the amount of make-up-removing oil(s) represents from 1 to 100% of the total weight of the oily phase.

33. The composition according to claim 1, wherein the make-up-removing oil is selected from the group consisting of hydrocarbonaceous oils, fatty esters comprising at least 8 carbon atoms, and their mixtures.

34. The composition according to claim 1, wherein the make-up-removing oil is selected from the group consisting of liquid paraffins, which may or may not be volatile, and their derivatives, liquid petrolatum, polydecenes, hydrogenated polyisobutene, such as parleam oil, hydrogenated isoparaffin, isohexadecane, isododecane, the esters obtained from a fatty acid with a straight or branched chain comprising at least 9 carbon atoms and from alcohols with a straight or branched chain comprising from 1 to 17 carbon atoms; the esters obtained from an acid with a straight or branched chain comprising 1 to 17 carbon atoms and from a fatty alcohol with a straight or branched chain comprising at least 9 carbon atoms; the esters obtained from benzoic acid and from a fatty alcohol with a straight or branched chain comprising at least 9 carbon atoms; and their mixtures.

35. The composition according to claim 1, wherein the oily phase additionally comprises at least one silicone oil.

36. The composition according to claim 1, wherein the amount of make-up-removing oil(s) represents from 10 to 80% of the total weight of the oily phase.

37. The composition according to claim 1, further comprising one or more adjuvants selected from the group consisting of hydrophilic or lipophilic active principles, preservatives, antioxidants, fragrances, solvents, fillers, fibres, sunscreen agents, colouring materials, polymers, basic or acidic agents, lipid vesicles, and their mixtures.

38. A method for removing make-up from and/or cleaning the skin, lips and/or eyes, comprising applying the composition of claim 1 to the skin, lips and/or eyes.

39. A cosmetic composition comprising an oily phase dispersed in an aqueous phase, and at least one amphiphilic polymer comprising at least one polymerized noncrosslinked monomer comprising ethylenic unsaturation comprising a sulphonic group, in the free or partially or completely neutralized form, comprising at least two hydrophobic side parts, wherein the oily phase comprises at least one make-up-removing oil and constitutes at least 40% by weight with respect to the total weight of the composition.

40. The composition according to claim 39, wherein the amount of oily phase ranges from 40 to 80% by weight with respect to the total weight of the composition.
